(19) United States
(12) Patent Application Publication
(10) Pub. No.: US 2022/0049245 A1
(43) Pub. Date: Feb. 17, 2022

(54) QUANTITATIVE MAPPING OF CHROMATIN ASSOCIATED PROTEINS

(71) Applicant: EPICYPHER, INC., Durham, NC (US)

(72) Inventors: Martis W. Cowles, Chapel Hill, NC (US); Zu Wen Sun, Brentwood, TN (US); Michael-Christopher Keogh, Cambridge, MA (US)

(21) Appl. No.: 17/430,741

(22) PCT Filed: Feb. 14, 2020
(86) PCT No.: PCT/US2020/018216

§ 371 Date: Aug. 13, 2021

Related U.S. Application Data
(60) Provisional application No. 62/806,174, filed on Feb. 15, 2019.

(51) Int. Cl. C12N 15/10; C07K 19/00; G01N 33/68

(52) U.S. Cl. CPC C12N 15/1065; G01N 2800/52; G01N 33/6875; C07K 19/00

(57) ABSTRACT

The present invention relates to DNA-barcoded recombinant nucleosomes and polynucleosomes that have been engineered for use as spike-in controls for the quantitative mapping of chromatin associated proteins using Chromatin ImmunoPrecipitation (ChIP) assays, tethered enzyme-based mapping assays, and other chromatin mapping assays. The invention further relates to methods of using the engineered DNA-barcoded recombinant nucleosomes in ChIP assays, tethered enzyme-based mapping assays, and other chromatin mapping assays.

### STATEMENT OF PRIORITY

This application claims the benefit of U.S. Provisional Application Ser. No. 62/806,174, filed Feb. 15, 2019, the entire contents of which are incorporated by reference herein.

### FIELD OF INVENTION

The present invention relates to DNA-barcoded recombinant nucleosomes and polynucleosomes engineered as spike-in controls for the quantitative mapping of chromatin associated proteins using chromatin immunoprecipitation (ChIP) assays, tethered enzyme-based mapping assays, and other chromatin mapping assays. The invention further relates to methods of using the engineered DNA-barcoded recombinant nucleosomes in ChIP assays, tethered enzyme-based mapping assays, and other chromatin mapping assays.

### BACKGROUND OF INVENTION

Chromatin Immunoprecipitation followed by next-generation sequencing (ChIP-seq) is widely used to map the genomic location of chromatin elements, such as histone post-translational modifications (PTMs) and chromatin associated proteins (ChAPs; e.g., transcription factors (TFs) or chromatin binding proteins (CBPs) (Collas 2010, Nakato and Shirahige 2017). In this approach, specific antibodies (or analogous affinity reagents) are used to enrich chromatin fragments containing specific PTMs or ChAPs. The associated DNA is then isolated and quantified using next-generation sequencing (NGS) or qPCR, respectively providing a genome-wide or local view of the target under study. ChIP-Seq has become a fundamental strategy to dissect genomic function and plays an essential role in drug target identification/pre-clinical drug validation studies. However, the approach is hampered by poor yields and low accuracy/reliability. Such limitations stem from the use of poorly validated ChIP-grade antibodies (Bock, Dhayalan et al. 2011, Egelhofer, Minoda et al. 2011, Fuchs, Krajewski et al. 2011, Fuchs and Strahl 2011, Nishikori, Hattori et al. 2012, Rothbart, Lin et al. 2012, Hattori, Taft et al. 2013, Rothbart, Dickson et al. 2015, Shah, Grzybowski et al. 2018), the inevitable background when enriching/amplifying specific regions from a vast excess of fragmented competitor chromatin, and the lack of internal controls capable of monitoring variability during chromatin enrichment/quantifying signals at target loci (Chen, Hu et al. 2015). Of note, exogenous xeno-chromatin (typically from yeast or Drosophila) has been employed as a spike-in control for sample normalization (Orlando, Chen et al. 2014, Egan, Yuan et al. 2016); however, natural chromatin as a reagent is poorly defined, and thus highly variable by many performance metrics. Moreover, this approach provides no insight to on-target antibody enrichment, leaving a significant need for quantitative mapping tools for ChAP targets, including TFs and other CBPs.

Alternative chromatin mapping methods have been developed beyond ChIP, including those that tether enzymes to genomic regions, resulting in release, enrichment, and subsequent analysis of target material (e.g., DamID, ChIC, ChEC, CUT&RUN, and CUT&Tag). For example, the related ChIC (Chromatin ImmunoCleavage (Schmid, Durussel et al. 2004)) and CUT&RUN (Cleavage Under Targets & Release Using Nuclease (Skene and Henikoff 2017, Skene, Henikoff et al. 2018)) methods use a factor-specific antibody to tether a fusion of protein A-Micrococcal Nuclease (pA-MNase) to genomic binding sites in intact cells or extracted nuclei, which is then activated by calcium addition to cleave DNA. pA-MNase provides a cleavage tethering system for antibodies to any PTM or ChAP. The CUT&RUN protocol is further streamlined by using a solid support (e.g., lectin-coated magnetic beads) to adhere cells (or nuclei). These advances simplify processing, increase sample recovery, and enable protocol automation. It is important to note that CUT&RUN assays are incredibly sensitive, requiring >100-fold less input material (i.e., cells) and 10- to 100-fold less sequencing depth than ChIP-Seq for selected PTMs (e.g., H3K27me3 (Skene and Henikoff 2017)) or transcription factors (e.g., CTCF [19]). Similar to CUT&RUN, CUT&Tag uses antibodies to bind chromatin proteins in situ, and then tethers a protein A and hyperactive Tn5 transposase (pA-Tn5) fusion to these sites. Upon controlled activation, the Tn5 selectively fragments and integrates adapter sequences at the genomic sites. The tagged target DNA is then amplified and sequenced, thereby bypassing several library preparation steps, saving time (total workflow time of <1 day) and eliminating a source of experimental bias. The high sensitivity (i.e., signal-to-noise) of the CUT&Tag approach make it amenable to ultra-low inputs, including single cell (Kaya-Okur, Wu et al. 2019).

Spike-in standards are essential for genome-wide analyses as they: i) are vital for normalization to enable cross-sample comparisons; and ii) can be used as internal controls to monitor assay performance (e.g., antibody specificity or technical variability). DNA-barcoded recombinant nucleosomes carrying defined histone PTMs were recently developed as spike-in controls to standardize ChIP methodology (named Internally Calibrated ChIP or ICeChIP; WO2015117145A1). A version of the ICeChIP approach has been commercialized under the SNAP-ChIP® spike-in platform. ICeChIP technology utilizes pools of DNA-barcoded dNucs carrying specific histone PTMs as internal standards to monitor antibody performance (i.e., specificity/efficiency and technical variability in situ) and for quantitative sample normalization. In this approach, DNA-barcoded nucleosome panels, comprised of one or more nucleosomes carrying unique PTMs at a single or range of concentrations(s), are spiked into samples before or after chromatin fragmentation. The resulting nucleosome mix (dNuc and cell derived) is immunoprecipitated with a bead-immobilized antibody specific for the PTM of interest. After subsequent processing, qPCR (or NGS) data from the IP and INPUT pools is analyzed for the number of reads detected for: 1) each DNA barcode; and, 2) sample DNA. Read numbers for each IP are then normalized to the INPUT concentration for each barcoded dNuc, providing a direct quantitation of sample DNA reads. dNucs serve as direct performance reagents/calibrators as they mimic the endogenous antibody target (modified mononucleosomes) and are subject to the same sources of variability experienced by the sample chromatin during ChIP processing. This technology was recently used to systematically examine the specificity of antibodies that target various methylforms of H3K4 (e.g., me1, me2, or me3) (Shah, Grzybowski et al. 2018).

In addition to ChIP methodology, DNA-barcoded recombinant nucleosomes have also been applied to develop medium-throughput chromatin binding ((Nguyen, Bittova et al. 2014); WO 2013/184930) and remodeling (Dann, Liszczak et al. 2017) assays. In each application, DNA-barcoded nucleosomes are comprised of synthetic DNA template encoding a unique ‘identifier sequence’ (or ‘barcode’) wrapped around a histone octamer carrying one or more PTM(s). For genomic mapping, DNA-barcoded nucleosomes can be pooled at one or more concentrations to represent multiple related marks for antibody specificity testing or ChIP assay normalization. However current DNA-barcoded recombinant nucleosomes cannot be applied to genomic mapping studies (e.g., ChIP, ChIC, CUT&RUN, or CUT&Tag) for ChAPs as they lack the epitopes required for representative antibody capture.

Given the above, there is a need in the art for improved controls for chromatin assays, such as ChIP assays and chromatin mapping assays using tethered enzymes.

### SUMMARY OF INVENTION

The present invention relates to the development and application of DNA-barcoded recombinant nucleosomes as spike-in controls for ChAP mapping studies. ChAPs include any protein that directly interacts with chromatin, including transcription factors that bind directly to DNA and ‘reader’ proteins/enzymes that interact with and/or modify histones and/or DNA. ChAPs also include proteins that indirectly interact with chromatin via macromolecular complexes, e.g., transcriptional regulation and chromatin remodeling complexes. Key changes to DNA-barcoded nucleosomes described in the prior art, include: a ChAP capture epitope such as 1) a ChAP epitope; or 2) a Short Peptide Tag (SPT; e.g., FLAG) fused to the N- or C-terminus of one of the histone subunits (e.g., histone H3, H4, H2A, or H2B) to capture ChAP- or SPT-specific antibodies for chromatin mapping studies (e.g., ChIP, ChIC, or CUT&RUN). The ChAP epitope may be an antibody binding sequence present in the ChAP being measured. The SPT may be one that has been added to the ChAP being measured. These spike-in controls may be used in several application formats, including assay optimization, antibody specificity testing, technical variability monitoring, and quantitative normalization for cross-sample comparisons.

In some embodiments, the ChAP capture epitope can be fused to the N- or C-terminus of a histone subunit (e.g., histone H3, H4, H2A, or H2B). In some embodiments, the ChAP capture epitope can replace a segment of a histone subunit. In some embodiments, the ChAP-histone protein can be recombinantly expressed as a fusion protein. In some embodiments, the ChAP capture epitope or histone protein can be chemically synthesized. In some embodiments, the ChAP-histone fusion protein can be generated by chemical or enzymatic linkage methods. These fusion linkages can be generated using two recombinant proteins, a synthetic peptide and a recombinant protein, or two synthetic peptides. Thus, ChAP-histone fusion proteins may be fully recombinant, semi-synthetic, or fully synthetic.

In some embodiments, DNA-barcoded nucleosomes containing a ChAP capture epitope can be used as spike-in controls for chromatin immunoprecipitation assays (i.e., ChIP, ChIP-qPCR, and ChIP-Seq). In some embodiments, these nucleosomes are assembled with 147 bp DNA. In some embodiments, these nucleosomes are assembled with DNA longer than 147 bp; i.e., comprising ‘linker’ DNA that extends beyond the nucleosome core particle (NCP). For example, a nucleosome may contain about 10, 20, 30, 40, 50, 60, 70, 80, 90, or 100 bp DNA on either side of the 147 bp NCP. In some embodiments, linker DNA is longer on one side of the NCP than the other. For example, a nucleosome may contain a 20 bp linker at the 5′ end and a 60 bp linker at the 3′ end. In some embodiments, DNA can be further modified to contain a binding moiety at the 5′ or 3′ end. Various DNA-barcoded nucleosomes carrying one or more ChAPs can be pooled at a single or range of concentrations. This nucleosome pool can be spiked into a ChIP reaction prior to the IP step. Capture efficiency of the on-target DNA-barcoded nucleosome by qPCR or NGS can be used to determine antibody specificity (by comparing on-target vs. off-target capture) or for sample normalization (by comparing on-target nucleosome capture between biological samples). In some embodiments, samples can be comprised of cells, tissues, or biological fluids (e.g., blood, plasma, serum, spinal fluid, saliva, etc.). In some embodiments, DNA length and modifications may be incorporated to make it forward compatible with other chromatin mapping approaches, including ChIP, ChIC, CUT&RUN, and CUT&Tag.

In some embodiments, DNA-barcoded nucleosomes containing a ChAP capture epitope can be used as spike-in controls for chromatin tethering assays (e.g., ChIC, CUT&RUN, and CUT&Tag). In some embodiments, these nucleosomes are assembled with DNA longer than 147 bp; i.e., comprising ‘linker’ DNA that extends beyond the NCP. The length of this linker may be optimized for maximum enzyme activity. For example, CUT&RUN assays, which target MNase to antibody targeted chromatin for subsequence cleavage, may require a different linker length for optimal MNase cleavage vs. CUT&Tag assays, which target chromatin using the hyperactive transposase Tn5. A nucleosome may contain about 10, 20, 30, 40, 50, 60, 70, 80, 90, or 100 bp DNA on either side of the 147 bp NCP. In some embodiments, linker DNA is longer on one side of the nucleosome core particle than the other. For example, a nucleosome may contain a 20 bp linker at the 5′ end and a 60 bp linker at the 3′ end. In some embodiments, DNA can be further modified to contain a binding moiety at the 5′ or 3′ end. This binding moiety can be used to bind nucleosomes to a solid support. Various DNA-barcoded nucleosomes carrying one or more ChAPs can be pooled at a single or range of concentrations. This nucleosome pool can be spiked into a chromatin tethering reaction and bound to a solid support prior to the ChAP-antibody incubation step. Capture efficiency of the on-target DNA-barcoded nucleosome can be determined via qPCR or NGS and be used to determine antibody specificity (by comparing on-target vs. off-target capture) or for sample normalization (by comparing on-target nucleosome capture between biological samples). In some embodiments, samples can be comprised of cells, tissues, or biological fluids (e.g., blood, plasma, serum, spinal fluid, saliva, etc.). In some embodiments, DNA length and modifications may be incorporated to make it forward compatible with other chromatin mapping approaches, including ChIP, ChIC, CUT&RUN, and CUT&Tag.

These and other aspects of the invention are set forth in more detail in the description of the invention below.

### BRIEF DESCRIPTION, OF THE DRAWINGS

FIGS. 1A-1B show (A) Overview of verSaNuc on-nucleosome ligation strategy using modified peptides. (B) Schematic of how verSaNuc approach can be used to rapidly generate ChAP-CUT&RUN dNucs (SEQ ID NO: 9).

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of nucleosomes, and transiently and stably transfected cells. Such techniques are known to those skilled in the art. See, e.g., SAMBROOK et al., MOLECULAR CLONING: A LABORATORY MANUAL 4th Ed. (Cold Spring Harbor, N.Y., 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The term “consisting essentially of” as used herein in connection with a nucleic acid, protein means that the nucleic acid or protein does not contain any element other than the recited element(s) that significantly alters (e.g., more than about 1%, 5% or 10%) the function of interest of the nucleic acid or protein.

As used herein, the term “polypeptide” encompasses both peptides and proteins, unless indicated otherwise.

A “nucleic acid” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

By “substantially retain” a property, it is meant that at least about 75%, 85%, 90%, 95%, 97%, 98%, 99% or 100% of the property (e.g., activity or other measurable characteristic) is retained.

The term “synthetic” refers to a compound, molecule, or complex that does not exist in nature.

The term “DNA barcode” refers to a nucleic acid sequence that can be used to unambiguously identify a DNA molecule in which it is located. The length of the barcode determines how many unique sequences can be present in a library. For example, a 1 nucleotide (nt) barcode can code for 4 library members, a 2 nt barcode 16 variants, 3 nt barcode 64 variants, 4 nt 256 variants, 5 nt 1,024 variants and so on. The barcode(s) can be single-stranded (ss) DNA or double-stranded (ds) DNA or a combination thereof.

One aspect of the invention relates to a nucleosome comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode indicative of a chromatin associated protein (ChAP) capture epitope; and
   c. the ChAP capture epitope fused to the N- and/or C-terminal end of one or more of the histones, or anywhere in the DNA molecule.

The nucleosome positioning sequence (NPS) can be any NPS known in the art. Examples include, without limitation, the Widom 601 sequence and the 601.2 and 601.3 variants, the Lytechinus variegatus 5S rDNA sequence, and the MMTV LTR nucleosomes A and B sequences.

The ChAP may be, without limitation, a transcription factor, a chromatin reader, a histone/DNA modifying enzyme, or a chromatin regulatory complex. Examples of transcription factors include, without limitation, those listed at: en.wikipedia.org/wiki/List_of human_transcription_factors, incorporated by reference herein in its entirety. Examples of readers include, without limitation, BRD4, YEATS2, and PWWP. Examples of histone/DNA modifying enzymes include, without limitation, NSD2, JMJD2A, CARM1, MLL1, DOT1L, EZH2, and DNMT3A/B. Examples of chromatin regulatory complexes include, without limitation, RNA Polymerase II, SMARCA2, and ACF.

The ChAP capture epitope may be any amino acid sequence that is present in the ChAP of interest and can be specifically bound by an antibody or other recognition or binding agent.

In some embodiments, the ChAP capture epitope is one or more short peptide tags. Examples of short peptide tags include, without limitation, FLAG (DYKDDDDK (SEQ ID NO: 1)), HA (YPYDVPDYA (SEQ ID NO: 2)), 6His (HHHHHH (SEQ ID NO: 3)), Myc (EQKLISEEDL (SEQ ID NO: 4)), Strep-I (AWRHPQFGG (SEQ ID NO: 5)), Strep-II (NWSHPQFEK (SEQ ID NO: 6)), protein C (EDQVDPRLIDGK (SEQ ID NO: 7)), V5, or GST or 2, 3, 4 or more repeats of the tags.

In some embodiments, the ChAP capture epitope is an antibody binding sequence, i.e., an epitope recognized and specifically bound by an antibody or other recognition or binding agent. In some embodiments, the epitope is one that is unique to the ChAP, e.g., having low sequence homology with related proteins (i.e., family members). In some embodiments, the epitope is one recognized by known antibodies to the ChAP.

Each of the histones in the nucleosome is independently fully synthetic (e.g., chemically synthesized), semi-synthetic (e.g., produced recombinantly then synthetically altered, e.g., by chemically or enzymatically adding a peptide sequence), or recombinant.

The DNA molecule may comprise further elements. In some embodiments, the DNA molecule further comprises a binding member linked to the DNA molecule, wherein the binding member specifically binds to a binding partner. Examples of the binding member and its binding partner include, without limitation, biotin with avidin or streptavidin, a nano-tag with streptavidin, glutathione with glutathione transferase, an antigen/epitope with an antibody, polyhistidine with nickel, a polynucleotide with a complementary polynucleotide, an aptamer with its specific target molecule, or Si-tag and silica. In some embodiments, the binding member is linked to the 5′ end of the DNA molecule. In some embodiments, the binding member is linked to the 3′ end of the DNA molecule.

In some embodiments, the DNA molecule comprises a linker between the nucleosome positioning sequence and the binding member that is about 10 to about 80 nucleotides in length, e.g., about 15 to about 40 nucleotides in length or about 15 to about 30 nucleotides in length, e.g., about 10, 15, 20, 25, 30, 35, 40, 45, 50, 55, 60, 65, 70, 75, or 80 nucleotides in length or any range therein.

In some embodiments, the DNA molecule comprises a nuclease or transposase recognition sequence, e.g., in the linker.

The nuclease or transposase recognition sequence may be any nucleotide sequence that is preferably recognized by a nuclease or transposase. In some embodiments, the nuclease or transposase recognition sequence is recognized by an endodeoxyribonuclease. Suitable endodeoxyribonucleases include, without limitation, micrococcal nuclease (MNase), Si nuclease, mung bean nuclease, pancreatic DNase I, yeast HO or I-SceI endonuclease, a restriction endonuclease, or a homing endonuclease, and modified or enhanced versions thereof. In some embodiments, the recognition sequence is an A/T-rich region.

In some embodiments, the nuclease or transposase recognition sequence is recognized by a transposase. Suitable transposases include, without limitation, Tn5, Mu, IS5, IS91, Tn552, Ty1, Tn7, Tn/O, Mariner, P Element, Tn3, Tn1O, or Tn903, and modified or enhanced versions thereof, e.g., a mutated hyperactive transposase. Such modified transposases are known in the art. In some embodiments, the transposase is Tn5 or a modified Tn5, e.g., a hyperactive Tn5 comprising one or more of the mutations E54K, M56A, or L372P. In some embodiments, the recognition sequence is a G/C-rich region.

In some embodiments, the linker comprises both a nuclease recognition sequence (e.g., one or more patches of A/T rich sequences) and a transposase recognition sequence (e.g., one or more patches of G/C rich sequences) so that the nucleosomes of the invention can be used for multiple methods. An A/T rich region or G/C rich region is one that contains more than 50%, A/T bases or G/C bases, respectively, e.g., more than 50%, 55%, 60%, 65%, 70%, 75%, or 80%.

In some embodiments, the DNA barcode has a length of about 6 to about 50 basepairs, e.g., about 7 to about 30 basepairs or about 8 to about 20 basepairs. In some embodiments, the DNA barcode may have a length of less than 50, 45, 40, 35, 30, 25, 20, 15, or 10 nucleotides. In some embodiments, the DNA barcode may have a length of at least 6, 7, 8, 9, 10, 12, 14, 16, 18, 20, 22, 24, 26, 28, or 30 nucleotides.

Another aspect of the invention relates to a panel (e.g., a collection) of the nucleosomes of the invention, wherein the nucleosomes in the panel comprise a ChAP capture epitope at one or more concentrations in the panel and the DNA barcode of each nucleosome indicates the concentration at which that nucleosome is present in the panel.

In some embodiments, the panel comprises at least two nucleosomes comprising different ChAP capture epitopes. In some embodiments, each nucleosome comprising a different ChAP capture epitope is present at the same concentration in the panel. In other embodiments, each nucleosome comprising a different ChAP capture epitope is present at multiple concentrations in the panel and the DNA barcode of each indicates that concentration at which that nucleosome is present in the panel.

In some embodiments, the panel may further comprise a synthetic nucleosome which does not comprise a ChAP capture epitope, e.g., as a control.

A further aspect of the invention relates to a polynucleosome comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode indicative of a ChAP capture epitope; and
c. the ChAP capture epitope fused to the N- and/or C-terminal end of one or more of the histones, or anywhere in the DNA molecule.

In some embodiments, the polynucleosome may comprise 2-10 nucleosomes, e.g., 2, 3, 4, 5, 6, 7, 8, 9, or 10 nucleosomes or any range therein.

The nucleosome positioning sequence (NPS) can be any NPS known in the art. Examples include, without limitation, the Widom 601 sequence and the 601.2 and 601.3 variants, the Lytechinus variegatus 5S rDNA sequence, and the MMTV LTR nucleosomes A and B sequences.

The ChAP capture epitope may be any amino acid sequence that is present in the ChAP of interest and can be specifically bound by an antibody or other binding agent.

In some embodiments, the ChAP capture epitope is one or more short peptide tags. Examples of short peptide tags include, without limitation, FLAG (DYKDDDDK (SEQ ID NO: 1)), HA (YPYDVPDYA (SEQ ID NO: 2)), 6His (HHHHHH (SEQ ID NO: 3)), Myc (EQKLISEEDL (SEQ ID NO: 4)), Strep-I (AWRHPQFGG (SEQ ID NO: 5)), Strep-II (NWSHPQFEK (SEQ ID NO: 6)), protein C (EDQVDPRLIDGK (SEQ ID NO: 7)), V5, TY1, or GST or 2, 3, 4 or more repeats of the tags.

In some embodiments, the ChAP capture epitope is an antibody binding sequence, i.e., an epitope recognized and specifically bound by an antibody.

Each of the histones in the nucleosome is independently fully synthetic (e.g., chemically synthesized), semi-synthetic (e.g., produced recombinantly then synthetically altered, e.g., by chemically or enzymatically adding a peptide sequence), or recombinant.

The DNA molecule may comprise further elements. In some embodiments, the DNA molecule further comprises a binding member linked to the DNA molecule, wherein the binding member specifically binds to a binding partner. Examples of the binding member and its binding partner include, without limitation, biotin with avidin or streptavidin, a nano-tag with streptavidin, glutathione with glutathione transferase, an antigen/epitope with an antibody, polyhistidine with nickel, a polynucleotide with a complementary polynucleotide, an aptamer with its specific target molecule, or Si-tag and silica. In some embodiments, the binding member is linked to the 5′ end of the DNA molecule. In some embodiments, the binding member is linked to the 3′ end of the DNA molecule.

In some embodiments, the DNA molecule comprises a linker between the nucleosome positioning sequence and the binding member that is about 10 to about 80 nucleotides in length, e.g., about 15 to about 40 nucleotides in length or about 15 to about 30 nucleotides in length, e.g., about 10, 15, 20, 25, 30, 35, 40, 45, 50, 55, 60, 65, 70, 75, or 80 nucleotides in length or any range therein.

In some embodiments, the DNA molecule comprises a nuclease or transposase recognition sequence, e.g., in the linker.

The nuclease or transposase recognition sequence may be any nucleotide sequence that is preferably recognized by a nuclease or transposase. In some embodiments, the nuclease or transposase recognition sequence is recognized by an endodeoxyribonuclease. Suitable endodeoxyribonucleases include, without limitation, micrococcal nuclease, Si nuclease, mung bean nuclease, pancreatic DNase I, yeast HO or I-SceI endonuclease, a restriction endonuclease, or a homing endonuclease, and modified or enhanced versions thereof. In some embodiments, the recognition sequence is an A/T-rich region.

In some embodiments, the nuclease or transposase recognition sequence is recognized by a transposase. Suitable transposases include, without limitation, Tn5, Mu, IS5, IS91, Tn552, Ty1, Tn7, Tn/O, Mariner, P Element, Tn3, Tn1O, or Tn903, and modified or enhanced versions thereof, e.g., a mutated hyperactive transposase. Such modified transposases are known in the art. In some embodiments, the transposase is Tn5 or a modified Tn5, e.g., a hyperactive Tn5 comprising one or more of the mutations E54K, M56A, or L372P. In some embodiments, the recognition sequence is a G/C-rich region.

In some embodiments, the linker comprises both a nuclease recognition sequence (e.g., one or more patches of A/T rich sequences) and a transposase recognition sequence (e.g., one or more patches of G/C rich sequences) so that the nucleosomes of the invention can be used for multiple methods. An A/T rich region or G/C rich region is one that contains more than 50%, A/T bases or G/C bases, respectively, e.g., more than 50%, 55%, 60%, 65%, 70%, 75%, or 80%.

In some embodiments, the DNA barcode has a length of about 6 to about 50 basepairs, e.g., about 7 to about 30 basepairs or about 8 to about 20 basepairs. In some embodiments, the DNA barcode may have a length of less than 50, 45, 40, 35, 30, 25, 20, 15, or 10 nucleotides. In some embodiments, the DNA barcode may have a length of at least 6, 7, 8, 9, 10, 12, 14, 16, 18, 20, 22, 24, 26, 28, or 30 nucleotides.

An additional aspect of the invention relates to an array comprising the polynucleosome of the invention. The polynucleosome array can contain a single ChAP capture epitope or be comprised of an ensemble of different ChAP capture epitopes. DNA barcodes on the array can be used to denote the entire array or unique features within the array.

A further aspect of the invention relates to a pool of the array of the invention, wherein each array comprises a unique ChAP capture epitope. In some embodiments, the polynucleosome array panel comprises, 2, 3, 4, 5, 6, 7, 8, 9, 10, 12, 15, 18, 21, 25, 30, 35, or 40 or more polynucleosome arrays comprising different ChAP capture epitopes or any range therein. In some embodiments, each polynucleosome array comprising a different ChAP capture epitope is present at the same concentration in the array. In other embodiments, each nucleosome array comprising a different ChAP capture epitope is present at multiple concentrations in the array and the DNA barcode of each polynucleosome indicates the concentration at which the polynucleosome is present in the array. In some embodiments, the array further comprises a polynucleosome array which does not comprise a ChAP capture epitope, e.g., for use as a control.

Another aspect of the invention relates to a solid support, e.g., a bead, comprising a binding partner to the binding member of the nucleosome, panel, polynucleosome, array, or pool of the invention, wherein the bead is bound to the nucleosome, panel, polynucleosome, array, or pool. The bead may be any bead suitable for separating chromatin, nucleosomes, or polynucleosomes from a sample and/or to attach the chromatin, nucleosomes, or polynucleosomes to a solid support. The bead may be composed of natural materials (e.g., alginate) or synthetic materials (e.g., polystyrene). In some embodiments, the bead is a magnetic bead that can be separated by exposure to a magnetic field.

An additional aspect of the invention relates to a kit comprising the nucleosome, panel, polynucleosome, array, pool, or bead of the invention. In some embodiments, the kit may further comprise an antibody, aptamer, nanobody, or other recognition or binding agent that specifically binds to a ChAP capture epitope or a nucleosome feature (e.g., histone post-translational modification (PTM), histone mutation, histone variant, or DNA post-transcriptional modification). In some embodiments, the kit may further comprise a nuclease or transposase linked to an antibody-binding protein or to an entity that binds the recognition agent. In certain embodiments, the antibody-binding protein may be, without limitation, protein A, protein G, a fusion between protein A and protein G, protein L, or protein Y. In some embodiments, the entity that binds the recognition agent is a protein. In other embodiments, the kit may further comprise a nuclease or transposase that is not linked to an antibody-binding protein or to an entity that binds the recognition agent. In certain embodiments, the kit may further comprise a bead comprising a binding partner to the binding member, e.g., a magnetic bead. The kit may further comprise reagents and/or containers for carrying out the methods of the invention, e.g., buffers, enzymes (e.g., nucleases, transposases, polymerases, ligases), detection agents, etc. In some embodiments, the kit may further comprise instructions for carrying out the methods of the invention.

The spike-in controls may be used in any chromatin assay known in the art in which an improved control/calibrator would be useful. Examples include, without limitation, the CUT&RUN assay (WO 2019/060907), the ChIC assay (U.S. Pat. No. 7,790,379), and the ICeChIP assay (WO 2015/117145). Each of these references are incorporated herein in their entirety.

One aspect of the invention relates to a method for chromatin mapping using tethered enzymes, wherein the improvement is the use of the nucleosome, panel, polynucleosome, array, pool, or bead of the invention in the assay as a spike-in control.

Another aspect of the invention relates to a method for mapping chromatin using tethered enzymes, comprising the steps of:
a) binding a nucleus, organelle, cell, or tissue to a solid support;
b) permeabilizing the nucleus, organelle, cell, or tissue;
c) binding the nucleosome, panel, polynucleosome, array, or pool of the invention to a solid support;
d) contacting the permeabilized nucleus, organelle, cell, or tissue of b) and the bound nucleosome, panel, polynucleosome, array, or pool of c) with an antibody, aptamer, nanobody, or recognition agent that specifically binds to the ChAP capture epitope;
e) adding an antibody-binding agent, aptamer-binding agent, nanobody-binding agent, or recognition agent-binding agent linked to a nuclease or transposase;
f) allowing the nuclease or transposase to cleave or label DNA in the nucleus, organelle, cell, or tissue and the nuclease or transposase recognition sequence in the nucleosome, panel, polynucleosome, array, or pool;
g) separating cleaved or labeled DNA; and
h) identifying the cleaved or labeled DNA; thereby mapping chromatin.

In some embodiments, the nuclease or transposase of step (e) is inactive and step (f) comprises activating the nuclease or transposase, e.g., by adding an ion such as calcium or magnesium.

In some embodiments, identifying the cleaved DNA comprises subjecting the cleaved DNA to amplification and/or sequencing. The sequencing may comprise, for example, qPCR, Next Generation Sequencing, or Nanostring.

In some embodiments, the method may further comprise determining the identity of the nucleosome, panel, polynucleosome, array, or pool based on the sequence of the DNA barcode in the cleaved or labeled DNA.

In some embodiments, the method further comprises optimizing the method based on the results detected with the nucleosome, panel, polynucleosome, array, or pool. For example, the recovery of on-target/off-target DNA-barcoded nucleosomes could be used to optimize enzyme concentration, enzyme activation time, cell-to-enzyme ratio, etc.

The methods may be carried out using any suitable format that provides a solid support for the cell, nucleus, organelle, or tissue. In some embodiments, the solid support is a bead, e.g., a magnetic bead. In some embodiments, the solid support is a well of a plate, e.g., 6, 12, 24, 96, 384, or 1536-well plates.

The results obtained from the methods of the invention may be used for any purpose where information on ChAPs and chromatin structure and/or modification, e.g., epigenetic changes, would be useful. In some embodiments, the methods may further comprise the step of using the sequencing results to compare chromatin features between healthy and disease tissues. In some embodiments, the methods may further comprise the step of using the sequencing results to predict a disease state. In some embodiments, the methods may further comprise the step of using the sequencing results to monitor response to therapy. In some embodiments, the methods may further comprise the step of using the sequencing results to analyze tumor heterogeneity.

The methods of the invention may be used for detecting and quantitating the presence of a ChAP on chromatin. An antibody, aptamer, nanobody, or recognition agent that specifically binds to a ChAP capture epitope may be used to detect and quantitate the ChAP at various genomic loci.

The methods of the invention may be used for determining and quantitating a ChAP on chromatin in a subject having a disease or disorder. An antibody, aptamer, nanobody, or recognition agent that specifically binds to a ChAP that may be associated with the disease or disorder of the subject or relevant to expression of a gene associated with the disease or disorder may be used to detect and quantitate the ChAP at various genomic loci. By this method, one can determine if a subject having a disease or disorder, e.g., a tumor, has a ChAP that is known to be associated with, e.g., the tumor type.

The methods of the invention may be used for monitoring changes in a ChAP on chromatin over time in a subject. This method may be used to determine if the status of the ChAP (e.g., level and/or activity) is improving, stable, or worsening over time. The steps of the method may be repeated as many times as desired to monitor changes in the status of a ChAP, e.g., 2, 3, 4, 5, 6, 7, 8, 9, 10, 25, 50, or 100 or more times. The method may be repeated on a regular schedule (e.g., daily, weekly, monthly, yearly) or on an as needed basis. The method may be repeated, for example, before, during, and/or after therapeutic treatment of a subject; after diagnosis of a disease or disorder in a subject; as part of determining a diagnosis of a disease or disorder in a subject; after identification of a subject as being at risk for development of a disease or disorder; or any other situation where it is desirable to monitor possible changes in the ChAP at various genomic loci.

The methods of the invention may be used for measuring on-target activity of a drug. The methods may be carried out before, during, and/or after administration of a drug to determine the capability of the drug to alter the ChAP status of the subject.

The methods of the invention may be used for monitoring the effectiveness of therapy in a subject having a disease or disorder. The steps of the method may be repeated as many times as desired to monitor effectiveness of the treatment, e.g., 2, 3, 4, 5, 6, 7, 8, 9, 10, 25, 50, or 100 or more times. The method may be repeated on a regular schedule (e.g., daily, weekly, monthly, yearly) or on as needed basis, e.g., until the therapeutic treatment is ended. The method may be repeated, for example, before, during, and/or after therapeutic treatment of a subject, e.g., after each administration of the treatment. In some embodiments, the treatment is continued until the method of the invention shows that the treatment has been effective.

The methods of the invention may be used for selecting a suitable treatment for a subject having a disease or disorder based on the ChAP status on chromatin in the subject. The methods may be applied, for example, to subjects that have been diagnosed or are suspected of having a disease or disorder. A determination of the ChAP status may indicate that the status of the ChAP has been modified and a therapy should be administered to the subject to correct the modification. Conversely, a determination that the status of the ChAP has not been modified would indicate that a therapy would not be expected to be effective and should be avoided.

The methods of the invention may be used for determining a prognosis for a subject having a disease or disorder based on the ChAP status on chromatin in the subject. In some instances, the ChAP is indicative of the prognosis of a disease or disorder. Thus, a determination of the ChAP status of an epitope in a subject that has been diagnosed with or is suspected of having a disease or disorder may be useful to determine the prognosis for the subject.

The methods of the invention may be used for identifying a biomarker of a disease or disorder based on the ChAP status on chromatin in a subject. In this method, biological samples of diseased tissue may be taken from a number of patients have a disease or disorder and the ChAP status determined. Correlations between the ChAP status and the occurrence, stage, subtype, prognosis, etc., may then be identified using analytical techniques that are well known in the art.

The methods of the invention may be used for screening for an agent that modifies the status of a ChAP on chromatin in a subject.

The screening method may be used to identify agents that increase or decrease the expression, level and/or activity of a ChAP. In some embodiments, the detected increase or decrease is statistically significant, e.g., at least p <0.05, e.g., p <0.01, 0.005, or 0.001. In other embodiments, the detected increase or decrease is at least about 10%, 20%, 30%, 40%, 50%, 60%, 70%, 80%, 90%, 100% or more.

Any compound of interest can be screened according to the present invention. Suitable test compounds include organic and inorganic molecules. Suitable organic molecules can include but are not limited to small molecules (compounds less than about 1000 Daltons), polypeptides (including enzymes, antibodies, and antibody fragments), carbohydrates, lipids, coenzymes, and nucleic acid molecules (including DNA, RNA, and chimeras and analogs thereof) and nucleotides and nucleotide analogs.

Further, the methods of the invention can be practiced to screen a compound library, e.g., a small molecule library, a combinatorial chemical compound library, a polypeptide library, a cDNA library, a library of antisense nucleic acids, and the like, or an arrayed collection of compounds such as polypeptide and nucleic acid arrays.

Any suitable screening assay format may be used, e.g., high throughput screening.

The method may also be used to characterize agents that have been identified as an agent that modifies the ChAP status on chromatin. Characterization, e.g., preclinical characterization, may include, for example, determining effective concentrations, determining effective dosage schedules, and measuring pharmacokinetics and pharmacodynamics.

In some embodiments, the nucleus, organelle, cell, or tissue is from a diseased tissue or sample. In some embodiments, the nucleus, organelle, cell, or tissue is from non-diseased tissue or sample. In some embodiments, the nucleus, organelle, cell, or tissue is or is from a peripheral tissue or cell, e.g., a peripheral blood mononuclear cell. In some embodiments, the nucleus, organelle, cell, or tissue is or is from cultured cells, e.g., primary cells.

One aspect of the invention relates to a method for assaying chromatin for a ChAP, wherein the improvement is the use of the nucleosome, panel, polynucleosome, array, pool, or bead of the invention in the assay as a spike-in control.

Another aspect of the invention relates to a method for quantifying the abundance of a chromatin associated protein (ChAP) in a biological sample using Chromatin ImmunoPrecipitation (ChIP), the method comprising:
a. isolating a biological sample;
b. preparing a library of native nucleosomes from the biological sample, wherein the library additionally comprises one or more ChAPs;
c. providing the nucleosome, panel, polynucleosome, array, pool, or bead of the invention comprising a ChAP capture epitope present in the ChAP to create a reference standard;
d. adding an antibody, aptamer, nanobody, or recognition agent that specifically binds to the ChAP capture epitope in the native nucleosome library and reference standard;
e. performing an affinity reagent-based assay to measure the amount of ChAP in the native nucleosome library and reference standard; and
f. quantifying ChAP abundance by comparing its relative abundance in the native nucleosome library to the reference standard.

An additional aspect of the invention relates to a method for quantifying the abundance of two or more ChAPs in a biological sample, the method comprising:
a. isolating a biological sample;
b. preparing a library of native nucleosomes from the biological sample, wherein the library comprises nucleosomes comprising two or more ChAPs;
c. providing the nucleosome, panel, polynucleosome, array, pool, or bead of the invention comprising ChAP capture epitopes present in the ChAPs to create a reference standard;
d. adding two or more antibodies, aptamers, nanobodies, or recognition agents that specifically bind to the ChAP capture epitopes to the native nucleosome library and the reference standard;
e. performing an affinity reagent-based assay to measure the amount of each ChAP in the native nucleosome library and the reference standard; and
f. quantifying the abundance of each ChAP by comparing the relative abundance in the native nucleosome library to the reference standard.

Another aspect of the invention relates to a method for quantifying the abundance of one or more ChAPs in a biological sample from a subject having a disease or disorder, the method comprising:
a. isolating a biological sample from the subject;
b. preparing a library of native nucleosomes from the biological sample, wherein the library comprises nucleosomes comprising one or more ChAPs;
c. providing the nucleosome, panel, polynucleosome, array, pool, or bead of the invention comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard; and
f. quantifying the abundance of ChAPs by comparing the relative abundance in the native nucleosome library to the reference standard.

A further aspect of the invention relates to a method for determining a prognosis for a subject having a disease or disorder based on the absolute quantification of one or more ChAPs, the method comprising:
a. isolating a biological sample from the subject;
b. preparing a library of native nucleosomes from the biological sample, wherein the library comprises nucleosomes comprising one or more ChAPs;
c. providing the nucleosome, panel, polynucleosome, array, pool, or bead of the invention comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP by comparing the relative abundance in the native nucleosome library to the reference standard; and
g. determining the prognosis of the subject based on the absolute abundance of the one or more ChAPs.

An additional aspect of the invention relates to a method for identifying a biomarker of a disease or disorder based on the absolute quantification of one or more ChAPs, the method comprising:
a. isolating a biological sample from the subject;
b. preparing a library of native nucleosomes from the biological sample, wherein the library comprises nucleosomes comprising one or more ChAPs;
c. providing the nucleosome, panel, polynucleosome, array, pool, or bead of the invention comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP by comparing the relative abundance in the native nucleosome library to the reference standard; and
g. correlating the absolute abundance of the one or more ChAPs with the disease or disorder; thereby identifying a biomarker of the disease or disorder.

Another aspect of the invention relates to a method of screening for an agent that modifies the ChAP status on chromatin from a biological sample of a subject, the method comprising determining the absolute quantification of one or more ChAPs in the presence and absence of the agent, wherein determining the absolute quantification of the one or more ChAPs comprises:
a. isolating a biological sample from the subject;
b. preparing a library of native nucleosomes from the biological sample, wherein the library comprises nucleosomes comprising one or more ChAP(s) in a target epitope(s);
c. providing the nucleosome, panel, polynucleosome, array, pool, or bead of the invention comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP by comparing the relative abundance in the native nucleosome library to the reference standard;
   wherein a change in the ChAP status in the presence and absence of the agent identifies an agent that modifies the ChAP status on chromatin.

The antibody, aptamer, nanobody, or recognition agent used in the methods of the invention may be any agent that specifically recognizes and binds to a ChAP of interest. In some embodiments, the affinity agent is an antibody or antibody fragment directed towards the ChAP capture epitope. The antibody or fragment thereof may be a full-length immunoglobulin molecule, an Fab, an Fab′, an F(ab)′₂, an scFv, an Fv fragment, a nanobody, a VHH or a minimal recognition unit. The agent may be an aptamer or a non-immunoglobulin scaffold such as an affibody, an affilin molecule, an AdNectin, a lipocalin mutein, a DARPin, a Knottin, a Kunitz-type domain, an Avimer, a Tetranectin or a trans-body. In some embodiments, the agent is an antibody or analogous enrichment reagent directed towards the ChAP capture epitope.

In some embodiments, the quantification of one or more ChAPs is determined by an affinity agent (e.g., antibody or analogous enrichment reagent)-based detection assay. Examples of antibody-based detection methods include, without limitation, ChIP, ELISA, AlphaLISA, AlphaSCREEN, Luminex, and immunoblotting. In some embodiments, the antibody-based detection assay uses two different antibodies for substrate capture and detection. In some embodiments, the antibody-based detection assay uses the same antibody for both substrate capture and detection.

In each of the methods of the invention, the biological sample may be any sample from which chromatin can be isolated. The biological sample may be, for example, blood, serum, plasma, urine, saliva, semen, prostatic fluid, nipple aspirate, lachrymal fluid, perspiration, feces, cheek swabs, cerebrospinal fluid, cell lysate samples, amniotic fluid, gastrointestinal fluid, biopsy tissue, lymphatic fluid, or cerebrospinal fluid. In some embodiments, the biological sample comprises cells and the chromatin is isolated from the cells. In some embodiments, the cells are cells from a disease of disorder associated with changes in one or more ChAPs, e.g., a diseased cell. In some embodiments, the cells are cells from a tissue or organ affected by a disease or disorder associated with changes in one or more ChAPs, e.g., a diseased tissue or organ. The cells may be obtained from the diseased organ or tissue by any means known in the art, including but not limited to biopsy, aspiration, and surgery. In some embodiments, the cells are cultured cells, e.g., primary cells.

In other embodiments, the cells are not cells from a tissue or organ affected by a disease or disorder associated with changes in ChAPs. The cells may be, e.g., cells that serve as a proxy for the diseased cells. The cells may be cells that are more readily accessible than the diseased cells, e.g., that can be obtained without the need for complicated or painful procedures such as biopsies. Examples of suitable cells include, without limitation, peripheral blood mononuclear cells.

In some embodiments, the biological sample is a biopsy. In other embodiments, the biological sample is a biological fluid. In some embodiments, the biological sample comprises peripheral blood mononuclear cells. In other embodiments, the biological sample comprises circulating nucleosomes, e.g., as released from dying cells. The circulating nucleosomes may be, e.g., from blood or from cells from a disease or disorder. In certain embodiments, the biological sample is plasma, urine, saliva, stool, lymphatic fluid, or cerebrospinal fluid. In some embodiments, the biological sample may be treated with an enzyme to digest chromatin into mono- and/or polynucleosomes. The enzyme may be, without limitation, a nuclease, e.g., micrococcal nuclease.

The subject may be any subject for which the methods of the present invention are desired. In some embodiments, the subject is a mammal, e.g., a human. In some embodiments, the subject is a laboratory animal, e.g., a mouse, rat, dog, or monkey, e.g., an animal model of a disease. In certain embodiments, the subject may be one that has been diagnosed with or is suspected of having a disease or disorder. In some embodiments, the subject may be one that is at risk for developing a disease or disorder, e.g., due to genetics, family history, exposure to toxins, etc.

Having described the present invention, the same will be explained in greater detail in the following examples, which are included herein for illustration purposes only, and which are not intended to be limiting to the invention.

### EXAMPLES

### Example 1

### Generation of ChAP-Containing Nucleosomes Using Recombinant Methods

Nucleosomes containing ChAP epitopes can be generated using any approach known in the art. Below we describe two methods. First, ChAP-histone fusion proteins can be directly expressed using recombinant methods. A series of nucleosomes (termed “verSaNuc”) were generated that contain common SPTs, including 3xFLAG, 3xTY1, and 3xHA. For these nucleosomes, the SPT followed by a GGGGS (SEQ ID NO: 8) linker fused to histone H3 was expressed. This modified histone was then incorporated into a recombinant nucleosome using 250 bp DNA (˜50 bp linker DNA on each side of the nucleosome core particle). A similar approach can be used for other ChAP fragments, such as CTCF. Second, ChAP-containing histones can be generated by linking synthetic peptides or recombinantly expressed proteins by chemical or enzymatic ligation.

Nucleosome spike-ins were engineered to contain a CTCF, BRD4 or 3xFLAG epitope (DYKDDDDK (SEQ ID NO: 1)) fused to the N-terminus of histone H3 to capture ChAP- or SPT-specific antibodies in genomic mapping assays (e.g., ChIP-seq, CUT&RUN, CUT&Tag). As an example, both ChAP epitopes and SPTs were selected that maximize user flexibility regarding antibody selection, enrichment strategy, and experimental design. Human CTCF (aa 650-727) and human BRD4 (aa 1031-1362) epitope regions were selected based on low sequence homology with related proteins (i.e., family members; C-terminal regions for both proteins) and contain the target epitope for the most widely used CTCF or BRD4 antibodies. To generate these nucleosomes, fusion histone proteins (e.g., CTCF-H3) were expressed in E. coli and purified, and then assembled into DNA-barcoded recombinant nucleosomes.

DNA-barcoded dNucs may be wrapped with a Widom 601 sequence (Lowary and Widom 1998) engineered with an embedded 22 bp barcode (composed of two catenated 11 bps) near the 3′ end (Herold, Kurtz et al. 2008), similar to spike-ins used for SNAP-ChIP spike-ins (e.g., EpiCypher K-MetStat; 19-1001). In SNAP-ChIP, spike-ins are assembled without ‘linker’ DNA (i.e., 147 bp). To make dNucs compatible with chromatin tethering technology (e.g., CUT&RUN/CUT&Tag), the DNA assembly sequence can be modified to include a 5′ biotin, which is used to immobilize the calibrators to a streptavidin-coated magnetic bead solid support. In addition, the nucleosome assembly sequence (i.e., 601 with embedded barcodes) was modified to include >20 bp linker DNA (i.e., DNA not wrapped around the histone octamer) to allow MNase to cleave and release the calibrator from the magnetic bead. Of note, MNase can reliably digest 15 bp linker regions in yeast and humans (Cole, Cui et al. 2016). To quality assess DNA-barcoded nucleosomes, they can be immobilized on beads and treated with MNase (digesting unassembled DNA) followed by qPCR to measure the nucleosome barcode sequence.

### Example 2

### Generation of ChAP-Containing Nucleosomes Using Enzyme Linkage

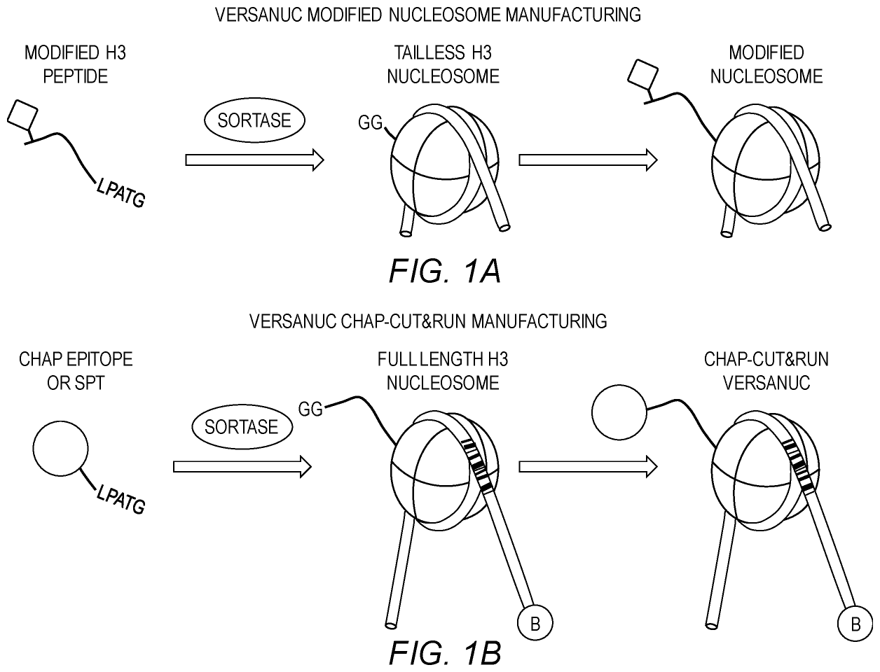

To accelerate nucleosome manufacturing, the S. aureus Sortase A (SrtA) transpeptidase can be used to ligate modified peptides directly onto fully assembled tailless nucleosomes (FIG. 1A). This approach delivers two capabilities: a) the rapid development of modified nucleosomes in small batches (μg vs. mg scale for standard dNuc assembly); and b) the multiplexing of modified nucleosome syntheses. This approach is very well-suited for ChAP-containing nucleosome development, which will require small quantities for each assay yet great diversity to meet market needs.

In one example, verSaNuc nucleosomes were assembled that contain: (i) a unique DNA barcode identifier, and (ii) a GGGGS (SEQ ID NO: 8) motif at the H3 N-terminus. Next, sortase-mediated on-nucleosome ligation reactions were performed using recombinant proteins (or synthetic peptides) encoding a ChAP epitope (or SPT) and a C-terminal native sortase target motif (LPATG (SEQ ID NO: 9); FIG. 1B).

Using this ‘on-nucleosome’ ligation approach, ChAP-containing nucleosome standards were generated for a set of ChAP epitopes and SPTs. Here, ChAP epitopes were focused on the BET family of bromodomain-containing proteins, including BRD2, BRD3, and BRD4. To generate this nucleosome panel, a C-terminal fragment of each protein was used, since this is divergent across the protein family and thus used for Ab development. ChAP epitopes were then generated by recombinant expression in E. coli. Each of these recombinant proteins were then ligated to a nucleosome containing a unique DNA-barcode. These nucleosomes can then be pooled, and doped into ChIP or chromatin tethering experiments, and used for antibody specificity testing, assay optimization, technical variability monitoring, or sample normalization.

The foregoing examples are illustrative of the present invention and are not to be construed as limiting thereof. Although the invention has been described in detail with reference to preferred embodiments, variations and modifications exist within the scope and spirit of the invention as described and defined in the following claims.

### REFERENCES

Bock, I., A. Dhayalan, S. Kudithipudi, O. Brandt, P. Rathert and A. Jeltsch (2011). “Detailed specificity analysis of antibodies binding to modified histone tails with peptide arrays.” Epigenetics 6(2): 256-263.
Chen, K., Z. Hu, Z. Xia, D. Zhao, W. Li and J. K. Tyler (2015). “The Overlooked Fact: Fundamental Need for Spike-In Control for Virtually All Genome-Wide Analyses.” Mol Cell Biol 36(5): 662-667.
Cole, H. A., F. Cui, J. Ocampo, T. L. Burke, T. Nikitina, V. Nagarajavel, N. Kotomura, V. B. Zhurkin and D. J. Clark (2016). “Novel nucleosomal particles containing core histones and linker DNA but no histone H1.” Nucleic Acids Res 44(2): 573-581.
Collas, P. (2010). “The current state of chromatin immunoprecipitation.” Mol Biotechnol 45(1): 87-100.
Dann, G. P., G. Liszczak, J. D. Bagert, M. M. Muller, U. T. T. Nguyen, F. Wojcik, Z. Z. Brown, J. Bos, T. Panchenko, R. Pihl, S. B. Pollock, K. L. Diehl, C. D. Allis and T. W. Muir (2017). “ISWI chromatin remodellers sense nucleosome modifications to determine substrate preference.” Nature.
Egan, B., C. C. Yuan, M. L. Craske, P. Labhart, G. D. Guler, D. Arnott, T. M. Maile, J. Busby, C. Henry, T. K. Kelly, C. A. Tindell, S. Jhunjhunwala, F. Zhao, C. Hatton, B. M. Bryant, M. Classon and P. Trojer (2016). “An Alternative Approach to ChIP-Seq Normalization Enables Detection of Genome-Wide Changes in Histone H3 Lysine 27 Trimethylation upon EZH2 Inhibition.” PLoS One 11(11): e0166438.
Egelhofer, T. A., A. Minoda, S. Klugman, K. Lee, P. Kolasinska-Zwierz, A. A. Alekseyenko, M. S. Cheung, D. S. Day, S. Gadel, A. A. Gorchakov, T. Gu, P. V. Kharchenko, S. Kuan, I. Latorre, D. Linder-Basso, Y. Luu, Q. Ngo, M. Perry, A. Rechtsteiner, N. C. Riddle, Y. B. Schwartz, G. A. Shanower, A. Vielle, J. Ahringer, S. C. Elgin, M. I. Kuroda, V. Pirrotta, B. Ren, S. Strome, P. J. Park, G. H. Karpen, R. D. Hawkins and J. D. Lieb (2011). “An assessment of histone-modification antibody quality.” Nat Struct Mol Biol 18(1): 91-93.
Fuchs, S. M., K. Krajewski, R. W. Baker, V. L. Miller and B. D. Strahl (2011). “Influence of combinatorial histone modifications on antibody and effector protein recognition.” Curr Biol 21(1): 53-58.
Fuchs, S. M. and B. D. Strahl (2011). “Antibody recognition of histone post-translational modifications: emerging issues and future prospects.” Epigenomics 3(3): 247-249.
Hattori, T., J. M. Taft, K. M. Swist, H. Luo, H. Witt, M. Slattery, A. Koide, A. J. Ruthenburg, K. Krajewski, B. D. Strahl, K. P. White, P. J. Farnham, Y. Zhao and S. Koide (2013). “Recombinant antibodies to histone post-translational modifications.” Nat Methods 10(10): 992-995.
Herold, J., S. Kurtz and R. Giegerich (2008). “Efficient computation of absent words in genomic sequences.” BMC Bioinformatics 9: 167.
Kaya-Okur, H. S., S. J. Wu, C. A. Codomo, E. S. Pledger, T. D. Bryson, J. G. Henikoff, K. Ahmad and S. Henikoff (2019). “CUT&Tag for efficient epigenomic profiling of small samples and single cells.” Nat Commun 10(1): 1930.
Lowary, P. T. and J. Widom (1998). “New DNA sequence rules for high affinity binding to histone octamer and sequence-directed nucleosome positioning.” J Mol Biol 276(1): 19-42.
Nakato, R. and K. Shirahige (2017). “Recent advances in ChIP-seq analysis: from quality management to whole-genome annotation.” Brief Bioinform 18(2): 279-290.
Nguyen, U. T., L. Bittova, M. M. Muller, B. Fierz, Y. David, B. IIouck-Loomis, V. Feng, G. P. Dann and T. W. Muir (2014). “Accelerated chromatin biochemistry using DNA-barcoded nucleosome libraries.” Nat Methods 11(8): 834-840.
Nishikori, S., T. Hattori, S. M. Fuchs, N. Yasui, J. Wojcik, A. Koide, B. D. Strahl and S. Koide (2012). “Broad ranges of affinity and specificity of anti-histone antibodies revealed by a quantitative peptide immunoprecipitation assay.” J Mol Biol 424(5): 391-399.
Orlando, D. A., M. W. Chen, V. E. Brown, S. Solanki, Y. J. Choi, E. R. Olson, C. C. Fritz, J. E. Bradner and M. G. Guenther (2014). “Quantitative ChlP-Seq normalization reveals global modulation of the epigenome.” Cell Rep 9(3): 1163-1170.
Rothbart, S. B., B. M. Dickson, J. R. Raab, A. T. Grzybowski, K. Krajewski, A. H. Guo, E. K. Shanle, S. Z. Josefowicz, S. M. Fuchs, C. D. Allis, T. R. Magnuson, A. J. Ruthenburg and B. D. Strahl (2015). “An Interactive Database for the Assessment of Histone Antibody Specificity.” Mol Cell 59(3): 502-511.
Rothbart, S. B., S. Lin, L. M. Britton, K. Krajewski, M. C. Keogh, B. A. Garcia and B. D. Strahl (2012). “Poly-acetylated chromatin signatures are preferred epitopes for site-specific histone H4 acetyl antibodies.” Sci Rep 2: 489.
Schmid, M., T. Durussel and U. K. Laemmli (2004). “ChIC and ChEC; genomic mapping of chromatin proteins.” Mol Cell 16(1): 147-157.
Shah, R. N., A. T. Grzybowski, E. M. Cornett, A. L. Johnstone, B. M. Dickson, B. A. Boone, M. A. Cheek, M. W. Cowles, D. Maryanski, M. J. Meiners, R. L. Tiedemann, R. M. Vaughan, N. Arora, Z. W. Sun, S. B. Rothbart, M. C. Keogh and A. J. Ruthenburg (2018). “Examining the Roles of H3K4 Methylation States with Systematically Characterized Antibodies.” Mol Cell.
Skene, P. J., J. G. Henikoff and S. Henikoff (2018). “Targeted in situ genome-wide profiling with high efficiency for low cell numbers.” Nat Protoc 13(5): 1006-1019.
Skene, P. J. and S. Henikoff (2017). “An efficient targeted nuclease strategy for high-resolution mapping of DNA binding sites.” Elife 6.

1. A nucleosome comprising:
a. a protein octamer, containing two copies each of histones H2A, H2B, H3, and H4, and optionally, linker histone H1;
b. a DNA molecule, comprising:
i. a nucleosome positioning sequence,
ii. a DNA barcode indicative of a chromatin associated protein (ChAP) capture epitope; and
c. the ChAP capture epitope fused to the N- and/or C-terminal end of one or more of the histones, or anywhere in the DNA molecule.

2. The nucleosome of claim 1, wherein the ChAP capture epitope is one or more short peptide tags. 3, (Original) The nucleosome of claim 2, wherein the one or more short peptide tags is FLAG (DYKDDDDK (SEQ ID NO: 1)), HA (YPYDVPDYA (SEQ ID NO: 2)), 6His (SEQ ID NO: 3)), Myc (EQKLISEEDL (SEQ ID NO: 4)), Strep-I (AWRHPQFGG (SEQ ID NO: 5)), Strep-II (NWSHPQFEK (SEQ ID NO: 6)), protein C (EDQVDPRLIDGK ((SEQ ID NO: 7)), V5, TY1, or GST or 2, 3, 4 or more repeats of the tags.

4. The nucleosome of claim 1, wherein the ChAP capture epitope is an antibody binding sequence.

5. (canceled)

6. The nucleosotne of claim 1, wherein the DNA molecule further comprises a binding member linked to the DNA molecule, wherein the binding member specifically binds to a binding partner.

7. The nucleosome of claim 6, wherein the DNA molecule comprises a linker between the nucleosome positioning sequence and the binding member that is about 10 to about 80 nucleotides in length.

8-9. (canceled)

10. The nucleosome of claim 6, wherein the DNA molecule comprises a nuclease or transposase recognition sequence.

11-16. (canceled)

17. The nucleosome of claim 6, wherein the binding member and its binding partner are biotin with avidin or streptavidin, a nano-tag with streptavidin, glutathione with glutathione transferase, an antigen/epitope with an antibody, polyhistidine with nickel, a polynucleotide with a complementary polynucleotide, an aptamer with its specific target molecule, or Si-tag and silica.

18-19. (canceled)

20. The nucleosome of claim 1, wherein the DNA barcode has a length of about 6 to about 50 basepairs.

21-22. (canceled)

23. A panel of the nucleosomes of claim 1, wherein the nucleosomes in the panel comprise a ChAP capture epitope at one or more concentrations in the panel and the DNA barcode of each nucleosome indicates the concentration at which that nucleosome is present in the panel.

24. A panel of the nucleosomes of claim 1, wherein the panel comprises at least two nucleosomes comprising different ChAP capture epitopes.

25-54. (canceled)

55. A kit comprising the nucleosome of claim 1.

56-60. (canceled)

61. A method for mapping chromatin using tethered enzymes, comprising the steps of:
a) binding a nucleus, organelle, cell, or tissue to a solid support;
b) permeabilizing the nucleus, organelle, cell, or tissue;
c) binding the nucleosome of claim 6 to a solid support;
d) contacting the permeabilized nucleus, organelle, cell, or tissue of b) and the bound nucleosome, panel, polynucleosome, array, or pool of c) with an antibody, aptamer, nanobody, or recognition agent that specifically binds to the ChAP capture epitope;
e) adding an antibody-binding agent, aptamer-binding agent, nanobody-binding agent, or recognition agent-binding agent linked to a nuclease or transposase;
f) allowing the nuclease or transposase to cleave or label DNA in the nucleus, organelle, cell, or tissue and the nuclease or transposase recognition sequence in the nucleosome, panel, polynucleosome, array, or pool;
g) separating cleaved or labeled DNA; and
h) identifying the cleaved or labeled DNA; thereby mapping chromatin.

62-79. (canceled)

80. A method for quantifying the abundance of a chromatin associated protein (ChAP) in a biological sample using Chromatin ImmunoPrecipitation (ChIP), the method comprising:
a. isolating a biological sample;
b. preparing a library of native nucleosomes from the biological sample, wherein the library additionally comprises one or more ChAPs;
c. providing the nucleosome of claim 1 comprising a ChAP capture epitope present in the ChAP to create a reference standard;
d. adding an antibody, aptamer, nanobody, or recognition agent that specifically binds to the ChAP capture epitope in the native nucleosome library and reference standard;
e. performing an affinity reagent-based assay to measure the amount of ChAP in the native nucleosome library and reference standard; and
f. quantifying ChAP abundance by comparing its relative abundance in the native nucleosome library to the reference standard.

81. A method for quantifying the abundance of two or more ChAPs in a biological sample, the method comprising:
a. isolating a biological sample;
b. preparing a library of native nucleosomes from the biological sample, wherein the library comprises nucleosomes comprising two or more core ChAP epitopes;
c. providing the nucleosome of claim 1 comprising ChAP capture epitopes present in the ChAPs to create a reference standard;
d. adding two or more antibodies, aptamers, nanobodies, or recognition agents that specifically bind to the ChAP capture epitopes to the native nucleosome library and the reference standard;
e. performing an affinity reagent-based assay to measure the amount of each ChAP in the native nucleosome library and the reference standard; and
quantifying the abundance of each ChAP by comparing the relative abundance in the native nucleosome library to the reference standard.

82. A method for quantifying the abundance of one or more ChAPs a biological sample from a subject having a disease or disorder, the method comprising:
a. isolating a biological sample from the subject;
b. preparing a library of native nucleosomes from the biological sample, wherein the library comprises nucleosomes comprising one or more ChAPs;
c. providing the nucleosome of claim 1 comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard; and
f. quantifying the abundance of ChAPs by comparing the relative abundance in the native nucleosome library to the reference standard.

83. A method for determining a prognosis for a subject having a disease or disorder based on the absolute quantification of one or more ChAPs, the method comprising:
a. isolating a biological sample from the subject;
b. preparing a library of native nucleosomes from the biological sample, wherein the library comprises nucleosomes comprising one or more ChAPs;
c. providing the nucleosome of claim 1 comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP in the target epitopes by comparing the relative abundance in the native nucleosome library to the reference standard; and
g. determining the prognosis of the subject based on the absolute abundance of the one or more ChAPs.

84. A method for identifying a biomarker of a disease or disorder based on the absolute quantification of one or more ChAPs, the method comprising:
a. isolating a biological sample from the subject;
b. preparing a library of native nucleosomes from the biological sample, wherein the library comprises nucleosomes comprising one or more ChAPs;
c. providing the nucleosome of claim 1 comprising ChAP capture epitopes present in the ChAPs to create a reference standard;
d. adding one or more antibodies, aptamers, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP by comparing the relative abundance in tie native nucleosome library to the reference standard; and
g. correlating the absolute abundance of the one or more ChAPs with the disease or disorder; thereby identifying a biomarker of the disease or disorder.

85. A method of screening for an agent that modifies the ChAP status on chromatin from a biological sample of a subject, the method comprising determining the absolute quantification of one or more ChAPs in the presence and absence of the agent, wherein determining the absolute quantification of the one or more ChAPs comprises:
a. isolating a biological sample from the subject;
b. preparing a library of native nucleosomes from the biological sample, wherein the library comprises nucleosomes comprising one or more ChAP(s) in a target epitope(s);
c. providing the nucleosome of claim 1 comprising ChAP capture epitopes present in the ChAPs to create a reference standard;
d. adding one or more antibodies, aptarners, nanobodies, or recognition agents that specifically bind to the ChAP capture epitopes to the native nucleosome library and the reference standard;
e. performing an affinity reagent-based assay to measure the amount of ChAP in the native nucleosome library and the reference standard;
f. quantifying the abundance of ChAP by comparing the relative abundance in the native nucleosome library to the reference standard;
wherein a change in the ChAP status in the presence and absence of the agent identifies an agent that modifies the ChAP status on chromatin.

86. The method of claim 80, wherein the biological sample comprises cells and the chromatin is isolated from the cells.

87-95. (canceled)


SEQUENCE LISTING (9 sequences)

SEQ ID NO: 1 - protein, 8 aa, Artificial
  peptide
DYKDDDDK

SEQ ID NO: 2 - protein, 9 aa, Artificial
  peptide
YPYDVPDYA

SEQ ID NO: 3 - protein, 6 aa, Artificial
  peptide
HHHHHH

SEQ ID NO: 4 - protein, 10 aa, Artificial
  peptide
EQKLISEEDL

SEQ ID NO: 5 - protein, 9 aa, Artificial
  peptide
AWRHPQFGG

SEQ ID NO: 6 - protein, 9 aa, Artificial
  peptide
NWSHPQFEK

SEQ ID NO: 7 - protein, 12 aa, Artificial
  peptide
EDQVDPRLIDGK

SEQ ID NO: 8 - protein, 5 aa, Artificial
  peptide
GGGGS

SEQ ID NO: 9 - protein, 5 aa, Artificial
  peptide
LPATG